(19) United States
(12) Patent Application Publication
(10) Pub. No.: US 2009/0042198 A1
(43) Pub. Date: Feb. 12, 2009

(54) METHOD FOR MEASURING CONCENTRATION OF NUCLEIC ACIDS

(75) Inventors: Rex Chin-Yih Hong, Taipei (TW); Herng-Er Horng, Taipei (TW); Hong-Chang Yang, Taipei (TW); Shieh-Yueh Yang, Taipei County (TW)

Correspondence Address: J C PATENTS, INC., 4 VENTURE, SUITE 250, IRVINE, CA, 92618, US

(73) Assignee: Shieh-Yueh Yang, Taipei County (TW)

(21) Appl. No.: 12/100,638

(22) Filed: Apr. 10, 2008

Related U.S. Application Data
(63) Continuation-in-part of application No. 11/867,207, filed on Oct. 4, 2007.
Application No. 11/867,207 is a continuation-in-part of application No. 11/563,035, filed on Nov. 24, 2006, now Pat. No. 7,394,246.
Application No. 11/563,035 is a continuation-in-part of application No. 11/422,336, filed on Jun. 6, 2006.
Application No. 11/422,336 is a continuation-in-part of application No. 11/164,275, filed on Nov. 16, 2005.

(51) Int. Cl. C12Q 1/68

(52) U.S. Cl. 435/6

(57) ABSTRACT

A method for measuring a concentration of nucleic acids is described. The method includes providing bioprobe molecules that include single-stranded nucleic acids, wherein the bioprobe molecules are conjugated with magnetic beads in a solution. A sample of single-stranded target nucleic acids is added to solution, where the single-stranded target nucleic acids hybridize with the single-stranded nucleic acids of the bioprobe molecules. A reduction of the ac (alternating current) magnetic susceptibility of the solution prior and after the addition of the sample to the solution is determined.

### CROSS-REFERENCE TO RELATED APPLICATION

This application is a continuation-in-part of prior application Ser. No. 11/867,207 filed on Oct. 4, 2007, now pending. The prior application Ser. No. 11/867,207 is a continuation-in-part of Ser. Nos. 11/563,035 and 11/422,336 filed on Nov. 24, 2006 and Jun. 6, 2006 respectively, both are pending now. And the prior application Ser. No. 11/422,336 is a continuation-in-part of prior application Ser. No. 11/164,275 filed on Nov. 16, 2005, now pending. The entirety of the above-mentioned patent application is hereby incorporated by reference herein and made a part of this specification.

### BACKGROUND OF THE INVENTION

1. Field of Invention

The present invention relates to a method for quantifying nucleic acids. More particularly, the present invention relates to a determination of the concentration of nucleic acids by magnetoreduction assay.

2. Description of Related Art

Conventionally, the concentration of nucleic acids, for example, DNAs (deoxyribonucleic acids) and RNAs (ribonucleic acids), are determined by gel electrophoresis and/or UV spectrophotometry. Nucleic acids, such as DNAs can be size-separated by gel electrophoresis and visualized by staining the gel with an ethidium bromide dye which binds to the nucleic acids. Nucleic acids can also be quantified in agarose gels by comparing with known standards through the measurements of the fluorescence emitted in ultra-violet light.

For a more accurate determination of the concentration of nucleic acids, UV spectrophotometry is commonly used. Both RNAs and DNAs absorb UV light very efficiently because the nitrogenous bases in the nucleotides have an absorption maximum at about 260 nm. Hence, it is possible to detect and quantify both RNAs and DNAs at very low concentrations. However, both methods have certain disadvantages.

The estimation of the concentrations of nucleic acids in a sample by gel electrophoresis is semi-quantitative and time-consuming, and the result could be confusing when numerous bands of DNA are observed. Further, ethidium bromide is toxic and carcinogenic; hence, it is undesirable to use. On the other hand, the accuracy of the UV method is easily affected by pH and ionic strength (salt concentration) of the buffer solution and is strongly relied on the calibration accuracy of the instrument. Further, a relatively large sample volume is required.

### SUMMARY OF THE INVENTION

In view of the foregoing, the present invention provides a methodology for measuring the concentration of nucleic acids.

According to the present invention, the method used is a magnetoreduction assay (MRA) in which the concentration of nucleic acids can be measured with high sensitivity.

As embodied and broadly described herein, a method for measuring the concentration of nucleic acids of the invention includes providing a magnetic reagent containing magnetic beads coated with bioprobe molecules; measuring a first alternating current (ac) magnetic susceptibility of the magnetic reagent; adding a sample containing target nucleic acids to the magnetic reagent, wherein the target nucleic acids become conjugated with the bioprobe molecules; measuring a second ac magnetic susceptibility of the magnetic reagent with the sample to determine the reduction in the ac magnetic susceptibility.

According to the method for measuring the concentration of nucleic acids of the present invention, wherein the target nucleic acids are single-stranded.

According to the method for measuring the concentration of nucleic acids of the present invention, each of the bioprobe molecules includes a single-stranded nucleic acid with a linker bond at an end of the single-stranded nucleic acid.

According to the method for measuring the concentration of nucleic acids of the present invention, the target single-stranded nucleic acids are complementary to the single-stranded nucleic acids of the bioprobe moleucles such that base-pairing reactions occur between the single-stranded nucleic acids of the bioprobe molecules and the single-stranded target nucleic acids prior to the measurement of the second (ac) magnetic susceptibility.

According to the method for measuring the concentration of nucleic acids of the present invention, the nucleic acids include DNAs (deoxyribonucleic acids) or RNAs (ribonuecleic acids).

According to the method for measuring the concentration of nucleic acids of the present invention, the magnetic beads are coated with a surfactant and the linker at each end of the single-stranded nucleic acid of the bioprobes are bonded with the surfactant on the magnetic beads.

According to the method for measuring the concentration of nucleic acids of the present invention, wherein an ac magnetic susceptibility reduction (Δ_{χac,φ}) between the first ac magnetic susceptibility _{χac,o} and the second ac magnetic susceptibility _{χac,φ} is determined and a parameter Δ_{χac,φ}≡(_{χac,o}−_{χac,φ}) is defined as an indicator of the concentration of the nucleic acids.

According to the method for measuring the concentration of nucleic acids of the present invention, wherein the ac magnetic susceptibility is measured using magnetic sensors including but not limited to SQUID (superconducting quantum interference device)-based magnetoreduction system.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. The patent or application file contains at least one drawing executed in color. Copies of this patent or patent application publication with color drawing(s) will be provided by the Office upon request and payment of the necessary fee.

FIG. 1 is a schematic diagram showing the structure of the magnetic reagent according to one embodiment of the invention.

FIG. 2 is a schematic diagram showing the structure of a bioprobe molecule according to an embodiment of the invention.

FIG. 3 is a schematic diagram showing the base-pairing between a bioprobe and a target DNA according to an embodiment of the invention.

FIG. 4 is a diagram showing a relationship between a reduction in ac magnetic susceptibility (Δ_{χac,φ}/_{χac,o}) and the concentration of single-stranded target DNA.

### DESCRIPTION OF THE PREFERRED EMBODIMENTS

The present invention provides an alternative methodology for measuring the concentration of nucleic acids. The method of the invention, the magnetoreduction assay (MRA), has been demonstrated to be capable of measuring the concentrations of biomolecules, including small biomolecules having a molecular weight of 23 Da or less, for example, with high sensitivity. The magentoreduction assay of the invention is performed using a SQUID-based MRA system, in which a superconducting quantum interference device is applied as the sensor of the MRA system. The details of the SQUID-based MRA system are disclosed in the priority U.S. patent application Ser. Nos. 11,867,207 and 11/563,035, which are incorporated herein by reference.

According to the method of measuring the concentrations of nucleic acids of the invention, a magnetic reagent is first provided. The magnetic reagent is a fluid containing homogeneously dispersed magnetic beads coated with a hydrophilic surfactant and bioprobe molecules attached to the exposed surface of the hydrophilic surfactant. The fluid is, for example, a phosphate buffer saline (PBS) solution. Each bioprobe molecule includes a nucleic acid bonded to a linker, for example, a single-stranded DNA (deoxyribonucleic acid) or a single-stranded RNA (ribonucleic acid) bonded to the linker at the phosphate group of the DNA or RNA, and the linker includes but not limited to a biotin molecule. In this embodiment, the bioprobe molecules that include single-stranded DNAs are used to demonstrate the essence and the efficacy of the present invention. The hydrophilic surfactant is, for example dextran, which serves to enhance the dispersion of the magnetic beads in the solution and the binding of the bioprobe molecules to the surfaces of the beads. It should be appreciated that other kinds of hydrophilic surfactant may also be applicable, for example, protein G, protein A, liposomes or organic acids. In this embodiment, the material of the core of the magnetic beads includes, for example, Fe₃O₄. However, other materials including MnFe₂O₄, Fe₂O₃, NiFe₂O₄ or CoFe₂O₄ are also applicable as the material of the magnetic beads and considered within the scope of the invention.

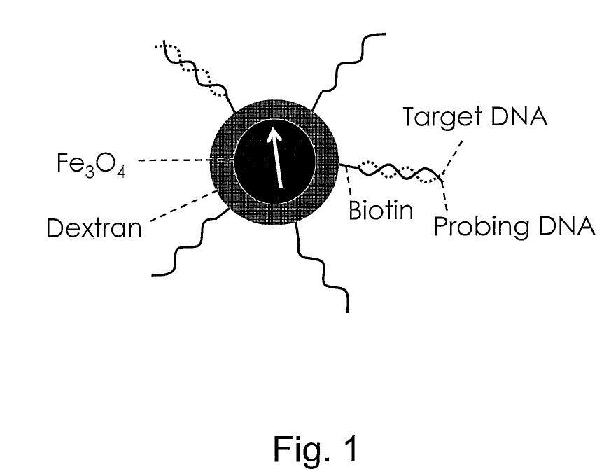

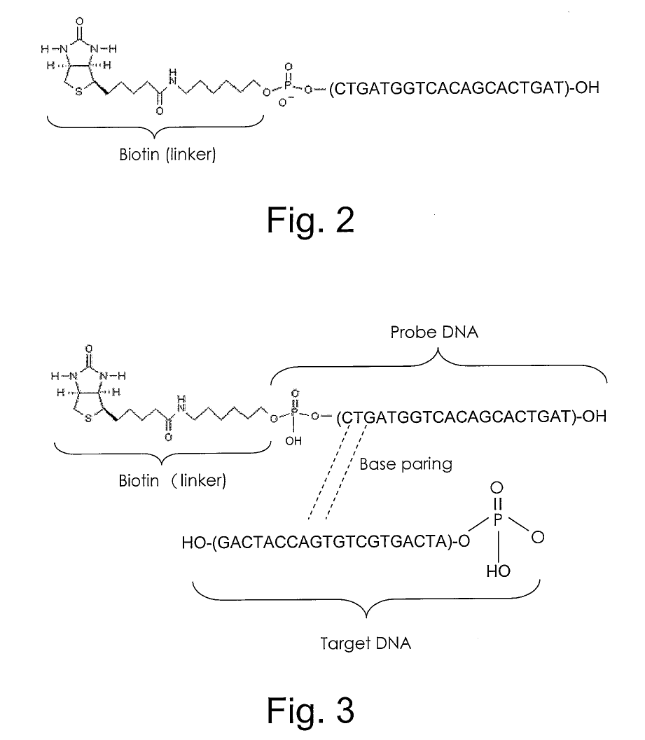

The preparation of the magnetic reagent principally includes dispersing magnetic beads in a phosphate buffer saline solution containing dextran (MagQu Co.) for coating the surfaces of the magnetic beads with the surfactant to form a magnetic fluid. In this embodiment, the concentration of the magnetic particles in the magnetic solution is about 0.3 emu/g, for example. Via oxidation, aldehyde groups (—CHO) are created on dextran coated on the surfaces of the magnetic beads. Thereafter, bioprobe molecules are added to the resulting magnetic fluid, and the aldehyde groups of dextran react with the linker at the end of the single-stranded nucleic acids to form bonds as shown in FIG. 2. In this embodiment, several tens of micro-grams of single-stranded DNA molecules are used. After the reaction between the bioprobe molecules and the magnetic beads is completed, which normally takes more than 12 hours, the unbounded single-stranded DNA molecules are separated from the solution through magnetic separation. The magnetic reagent with single-stranded DNA-coated magnetic beads, as shown in FIG. 1, is thereby obtained.

To evaluate the efficacy of the MRA on determining the concentrations of nucleic acids in a solution, a study on deoxyribonucleic acid (DNA) is performed. In this embodiment of the invention, various known concentrations of single-stranded target DNAs are prepared. The concentrations of the single-stranded target DNAs in this study vary from 10⁻⁴ to 10³×10⁻⁹ g/ml. Moreover, the single-stranded target DNAs are complementary to the single-stranded DNAs of the bioprobe molecules used in the study. In other words, the nucleotides of the target DNA molecules will hybridize with those of the DNA molecules of the bioprobe molecules. For example, two nucleotides on opposite complementary DNA strands are connected via hydrogen bonds, such that adenine (A) normally base-pairs with thymine (T), and cytosine (C) normally base-pairs with guanine (G), as shown in FIG. 3.

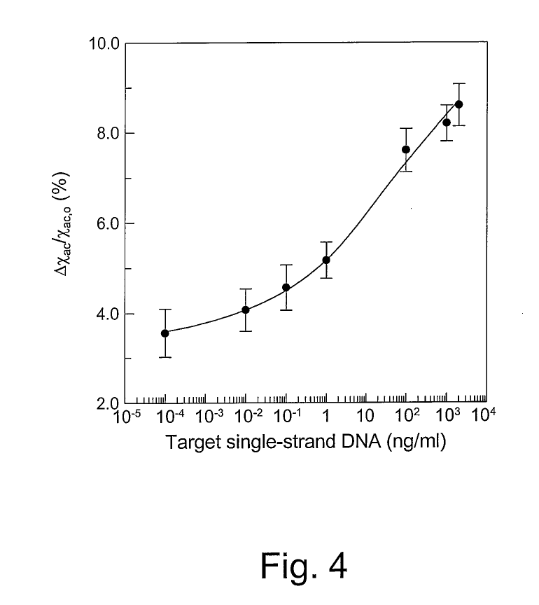

To perform the MRA measurement, the ac magnetic susceptibility spectrum of the magnetic reagent (_{χac,o}) is first determined using a SQUID-based magnetoreduction system, which basically includes at least two excitation coils driven by two independent function generators to provide the sample with two ac (alternating current) excitation magnetic fields to magnetize the sample, and the ac (alternating current) magnetic susceptibility signal of the sample is detected by a pick-up coil and is transferred to a SQUID magnetometer sensor to be analyzed by a spectrum analyzer. In this embodiment, about 100-μl of the reagent is mixed with 20-μ1 of a sample solution containing the single-stranded target DNA. Subsequent to an incubation period of 1 to 2 hours at room temperature for base-pairing to occur between the single-stranded target DNA and the single-stranded DNA on the bioprobes, the _{χac} spectrum of the resulting mixture solution (using a sample volume of 20 μl ) is then determined. A reduction in the _{χac} at a given frequency, for example tens Hz to tens kHz, is observed for each mixture solution containing a given concentration of the single-stranded target DNA. Hence, as shown in FIG. 4, a relationship between a reduction in ac magnetic susceptibility (for example, Δ_{χac,φ}/_{χac,o}) and the concentration of the single-stranded target DNAs is established, wherein Δ_{χac,φ}/_{χac,o} is defined as (_{χac,o}−_{χac,φ})/_{xac,o}. The details of the mechanisms and the theory behind the MRA are disclosed in the priority U.S. patent application Ser. Nos. 11/164,275 and 11/867,207, which are incorporated herein by reference.

As indicated by the results of the study in this embodiment of the invention shown in FIG. 4, the magnetoreduction assay is applicable in determining the concentrations of nucleic acids with high sensitivity. The results of the study indicate that the sensitivity of MRA in determining the concentration of nucleic acids, such as DNAs, is about 5 pg/ml and the sensitivity of MRA in determining the mass of nucleic acids, such as DNAs is about 0.1 pg. Moreover, the detectable range of the DNA concentration is greater than 6 orders of magnitude, for example, from 10⁻¹³ to 10⁻⁶ g/ml. Accordingly, the magentoreduction assay of the invention can provide an accurate quantitative determination of the concentrations of nucleic acids.

It will be apparent to those skilled in the art that various modifications and variations can be made to the structure of the present invention without departing from the scope or spirit of the invention. In view of the foregoing descriptions, it is intended that the present invention covers modifications and variations of this invention if they fall within the scope of the following claims and their equivalents.

What is claimed is:

1. A method of measuring a concentration of nucleic acids, the method comprising:
providing a magnetic reagent that comprises magnetic beads coated with bioprobe molecule;
measuring a first alternating current (ac) magnetic susceptibility of the magnetic reagent (_{χac,o});
adding a sample containing target nucleic acids to the magnetic reagent, wherein the target nucleic acids are conjugated with the bioprobe molecules;
measuring a second ac magnetic susceptibility of the magnetic reagent (_{χac,φ}) after the base-pairing reaction, so as to obtain an ac magnetic susceptibility reduction (Δ_{χac,φ}) between the first ac magnetic susceptibility and the second ac magnetic susceptibility, wherein Δ_{χac,φ}≡(_{χac,o}−_{χac,φ}).

2. The method of claim 1, wherein the target nucleic acids are single-stranded.

3. The method of claim 1, wherein each of the bioprobe molecules comprises a single-stranded nucleic acid.

4. The method of claim 1, wherein the single-stranded target nucleic acids are complementary to the single-stranded nucleic acids of the bioprobe molecules such that base-pairing reactions occur between the single-stranded target nucleic acids and the single-stranded nucleic acids of the bioprobe molecules.

5. The method of claim 1, wherein the magnetic beads are coated with a hydrophilic surfactant.

6. The method of claim 5, wherein each bioprobe molecule comprises a linker at an end of each single-stranded nucleic acid of the bioprobe molecules, and the linker is bonded to the hydrophilic surfactant coated on the magnetic beads.

7. The method of claim 5, wherein the hydrophilic surfactant comprises dextran, protein G, protein A, liposomes or organic acids.

8. The method of claim 1, wherein the nucleic acids comprise DNAs (deoxyribonucleic acids) or RNAs (ribonuecleic acids).

9. The method of claim 1, wherein the first alternating current (ac) magnetic susceptibility and the second alternating current (ac) magnetic susceptibility are measured using a SQUID (superconducting quantum interference device)-based magnetoreduction system.

10. The method of claim 1, wherein a parameter Δ_{χac,φ}/_{χac,o}≡(_{χac,o}−_{χac,φ})/_{χac,o} is defined as an indicator of the concentration of the nucleic acids.

11. The method of claim 1, wherein the first ac magnetic susceptibility of the magnetic reagent (_{χac,o}) and the second ac magnetic susceptibility of the magnetic reagent (_{χac,φ}) are determined at a frequency within a range from tens Hz to tens kHz.

12. The method of claim 1, wherein the nanoparticles are formed with a material selected from the group consisting of Fe₃O₄, Fe₂O₃, MnFe₃O₄, NiFeO₄ and CoFe₂O₄.
